(19) United States
(12) Patent Application Publication
(10) Pub. No.: US 2018/0250520 A1
(43) Pub. Date: Sep. 6, 2018

(54) SYSTEMS FOR SIGNALING A REMOTE TISSUE RESPONSIVE TO INTERACTION WITH ENVIRONMENTAL OBJECTS

(71) Applicant: ELWHA LLC, Bellevue, WA (US)

(72) Inventors: Roderick A. Hyde, Redmond, WA (US); Jordin T. Kare, San Jose, CA (US); Eric C. Leuthardt, St. Louis, MO (US); Mark A. Malamud, Seattle, WA (US); Tony S. Pan, Bellevue, WA (US); Elizabeth A. Sweeney, Seattle, WA (US); Clarence T. Tegreene, Mercer Island, WA (US); Charles Whitmer, North Bend, WA (US); Lowell L. Wood, JR., Bellevue, WA (US)

(21) Appl. No.: 15/450,765

(22) Filed: Mar. 6, 2017

(51) Int. Cl. A61N 2/00; A61B 5/11; A61B 5/00; A61N 2/02; A61F 7/00; A61N 7/00; A61H 23/00

(52) U.S. Cl. CPC A61N 2/006; A61H 2205/12; A61B 5/7455; A61N 2/02; A61F 7/00; A61N 7/00; A61H 23/00; A61B 5/6807; A61B 5/6849; A61B 5/7275; A61B 5/7282; A61B 5/746; A61B 2560/0242; A61B 2562/0204; A61B 2562/0219; A61B 2562/0233; A61B 2562/0247; A61B 2562/0257; A61B 2562/0261; A61B 2562/164; A61F 2007/0045; A61N 2007/0026; A61B 5/112

(57) ABSTRACT

A system embodiment includes, but is not limited to, a first device configured to interface with a first body portion, the first device including a sensor assembly configured to generate sense signals associated with an impact or an impending impact between the environmental object and the first body portion; and a reporting device configured to generate communication signals responsive to generation of the sense signals for remote transmission. The system also includes, but is not limited to, a processor to process at least one of the sense signals or the communication signals, and a second device configured to interface with a second body portion remote from the first body portion, the second device including a communications interface configured to receive the communication signals from the first device; and a stimulator configured to generate stimulation of a tissue remote the first body portion responsive to instruction by the processor.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a system includes, but is not limited to, a first device configured to interface with a first body portion of an individual, the first device including a first deformable substrate configured to conform to the first body portion; a sensor assembly coupled to the first deformable substrate, the sensor assembly including a sensor configured to generate one or more sense signals associated with an impact between an environmental object and the first body portion or an impending impact between the environmental object and the first body portion; and a reporting device operably coupled to the sensor assembly and configured to generate one or more communication signals responsive to generation of the one or more sense signals by the sensor assembly for remote transmission, the one or more communication signals associated with the impact between the environmental object and the first body portion or the impending impact between the environmental object and the first body portion. The system also includes, but is not limited to, a processor operably coupled to at least one of the sensor assembly or the reporter to process at least one of the one or more sense signals or the one or more communication signals; and a second device configured to interface with a second body portion of the individual remote from the first body portion, the second device including a second deformable substrate configured to conform to the second body portion; a communications interface coupled to the second deformable substrate and configured to receive at least one of the one or more communication signals from the first device or instructions from the processor and a stimulator operably coupled to the processor and configured to generate stimulation of a tissue of the second body portion remote from a site of impact between the environmental object and the first body portion or a site of potential impact between the environmental object and the first body portion responsive to instruction by the processor.

In an aspect, a method includes, but is not limited to, detecting, via a sensor positioned on a first body portion of an individual, an impact between an environmental object and the first body portion or an impending impact between the environmental object and the first body portion; generating one or more sense signals responsive to detecting the impact between the environmental object and the first body portion or the impending impact between the environmental object and the first body portion; transmitting the one or more sense signals to a processor; and stimulating a tissue with a second device positioned on a second body portion of the individual remote from the first body portion, where the tissue is remote from a site of impact between the environmental object and the first body portion or a site of potential impact between the environmental object and the first body portion responsive to instruction by the processor.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of a system for sensing an impact or potential impact between a body portion and an environmental object with a first device at a first location, transmitting information to a second device at a second location remote from the first location, and stimulating a tissue remote from a site of impact or potential impact at the first location.

FIG. 2A is a schematic of an embodiment of a system such as shown in FIG. 1.

FIG. 2B is a schematic of an embodiment of a system such as shown in FIG. 1.

FIG. 2C is a schematic of an embodiment of a system such as shown in FIG. 1.

FIG. 3 is a schematic of an embodiment of a system such as shown in FIG. 1.

FIG. 4A is a schematic of an embodiment of a system such as shown in FIG. 1.

FIG. 4B is a schematic of an embodiment of a system such as shown in FIG. 1.

FIG. 4C is a schematic of an embodiment of a system such as shown in FIG. 1.

FIG. 5 is a schematic of an embodiment of a system such as shown in FIG. 1.

FIG. 6 is a schematic of an embodiment of a system such as shown in FIG. 1.

FIG. 7 is a flowchart of a method of monitoring or detecting an interaction between a first body portion of an individual subject and an environmental object and for stimulating a tissue of a second body portion remote from the first body portion or from a location of the interaction.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Systems and methods are described for wearable devices for monitoring or detecting an interaction between a first body portion (e.g., a foot, a leg, a hand, etc.) of an individual subject and an environmental object and for stimulating a tissue of a second body portion (e.g., a back, a spine, a leg, an arm, etc.) remote from the first body portion or from a location of the interaction. The interaction can include an impact or an impending impact between the first body portion and the environmental object. The systems and methods described herein can facilitate coordination of movement or interpretation of nervous signals, or lack thereof, by an individual subject, such as by assisting in information transfer where nerves may be damaged (e.g., via neuropathy, injury, illness, etc.), providing the individual subject a signal to retrain actions in response to various stimuli (e.g., via a trained response to alter an interpretation of a nerve signal), assisting in proprioception, or the like. For example, an individual's proprioception, the sense of relative position of a body part that normally enables the individual to move with respect to his environment (e.g., to walk while avoiding stumbling or loss of balance, to pick up a pen without hitting the table, etc.) can be affected by peripheral neuropathy, leading to loss of coordination and loss of tissue with subsequent reduction in movement capability. Individuals afflicted with neuropathy (e.g., peripheral neuropathy) may have reduced capabilities to detect when a body portion comes in contact with another object or a surface due to an impairment of sensation, movement, or other normal body functions. Neuropathy can result from a systemic disease or disorder (e.g., diabetes or immune system disease), from an injury or disorder affecting the central nervous system (e.g., poststroke neuropathy or spasticity, or poststroke syndrome, Parkinson's disease, etc.) or one or more peripheral nerve (e.g., cancer or traumatic injury), from interactions with various medications or medical treatments (such as chemotherapy), from inherited characteristics, from vitamin deficiency, from traumatic injury, from excessive alcohol usage, from infections (e.g., human immunodeficiency virus (HIV) or herpes zoster (shingles)), from disrupted blood flow (e.g., by vasculitis, ischemia, or edema), or other conditions and sources. Neuropathy can involve a single nerve (mononeuropathy such as causing a palsy) or multiple nerves (polyneuropathy). Peripheral neuropathy can involve a distributed effect, where longer nerves are first affected, with distribution of effect from the distal nerve region. For instance, a foot or hand could be affected by neuropathy, whereby nerve function may be inhibited, and then a distribution of effect could occur, affecting the knee or elbow for example. Shorter nerves (e.g., nerves on the back) may not be affected by peripheral neuropathy, or be affected to a lesser degree than peripheral appendages, thus body portions having shorter nerves could serve as the second body portion to signal tissue relatively unaffected by nerve inhibition.

The systems and methods described herein can include a first device to interface with the first body portion and provide monitoring or detecting of an interaction between the first body portion and an environmental object. For instance, the first device can employ a deformable substrate, a sensor assembly, and a reporting device to monitor or detect an interaction between a first body portion of an individual subject and an environmental object, where such interaction can include a physical impact (e.g., contact) or impending impact (e.g., likely impact, estimated impact, inferred impact, etc.). The sensor assembly can include, but is not limited to, an accelerometer, a pressure sensor, a proximity sensor, a strain sensor, an acoustic sensor, an ultrasonic sensor, a radio frequency sensor, an optical sensor, an infrared sensor, a radar sensor, a sensor array, or combinations thereof. For example, the sensor assembly can sense an interaction between a foot and a furniture item or room surface (e.g., a floor, a wall, a doorframe, etc.), where such interaction can include a physical impact between the foot and the furniture item or room surface or an impending physical impact, such as inferred from movement, proximity, or the like of the first body portion or the environmental object. The deformable substrate of the first device can include wearable components including, but not limited to, a footgear structure, a structure to couple to existing footgear, an ankle cuff structure, an apparel clip structure, a substrate attachable to a skin surface, or combinations thereof. The systems and methods described herein also include a processor to process various information obtained from or transmitted to the components of the system, including but not limited to the first device and components thereof (e.g., the sensor assembly, the reporting device), a second device in communication with the first device, an external device, or the like. For example, the processor can receive information from the sensor assembly to determine whether a signal should be sent from the first device to the second device via the reporting device. In embodiments, the processor can control operation of the sensor assembly. For example, the processor can control operation of the sensor assembly based on feedback from the sensor assembly, feedback from the second device, on instructions from an external device (e.g., mobile communications device), or the like. The reporting device can be operably coupled to the sensor assembly to transmit information to the second device remote from the first device, such as on a second body portion. The information can include, but is not limited to, communication signals associated with an impact between the first body portion and the environmental object or communication signals associated with an impending impact between the first body portion and the environmental object.

The second device is remotely located from the first device via a deformable substrate coupled to a second body portion. The second device can include a communications interface to receive information from the first device, such as via wireless protocols, wired connections, or combinations thereof. The second device can also include a processor to receive and analyze information received from the first device, from an external device, or combinations thereof. The processor can control operation of a stimulator of the second device to facilitate stimulation of a tissue remote from the first body portion (e.g., remote from a site of impact between the environmental object and the first body portion or a site of potential impact between the environmental object and the first body portion). For example, the processor can receive information from the first device indicating when to activate the stimulator, when to deactivate the stimulator, or the like. For example, the processor can analyze communication signals from the first device or from an external device to determine when to activate the stimulator, when to deactivate the stimulator, or the like, such as by determining that an impact has occurred, determining a likelihood of impact, or the like. The stimulator can include, but is not limited to, an electrical stimulator, an electromagnetic stimulator, a thermal stimulator, an acoustic stimulator, a vibration stimulator, an ultrasonic stimulator, or combinations thereof. The tissue stimulated by the stimulator can include a specific nerve, a cutaneous tissue, cutaneous nerve, a peripheral nerve, or combinations thereof, where a type of stimulation can depend on a location of the tissue, depth of tissue, or the like. For example, cutaneous tissue can be stimulated via vibration stimulator, thermal stimulator, or combinations thereof; deeper tissues (e.g., nerves innervating muscle) can be stimulated via electrical stimulator (e.g., direct nerve stimulation with electrode). The stimulation can facilitate coordination of movement or interpretation of nervous signals, or lack thereof, by the individual, such as by assisting in information transfer where nerves may be damaged (e.g., via neuropathy, injury, illness, etc.), providing the individual subject a signal to retrain actions in response to various stimuli (e.g., via a trained response to alter an interpretation of a nerve signal), assisting in proprioception, or the like.

The systems and methods herein can be part of a therapeutic regimen, for example part of a physical therapy regimen, occupational therapy regimen, stroke therapy regimen, or other rehabilitation.

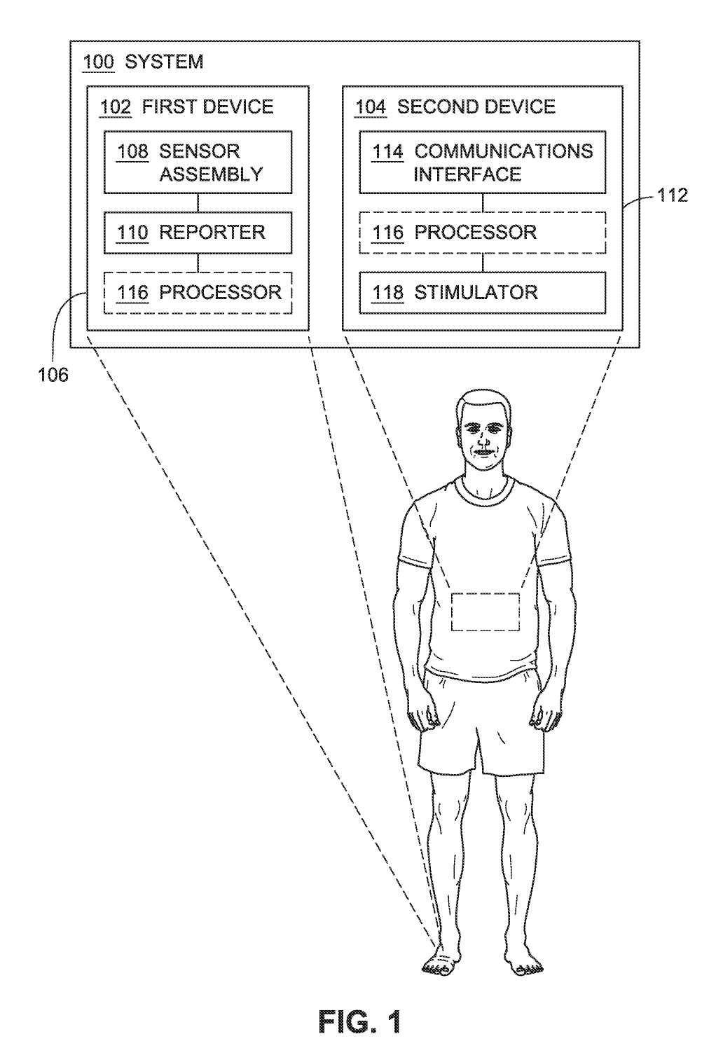

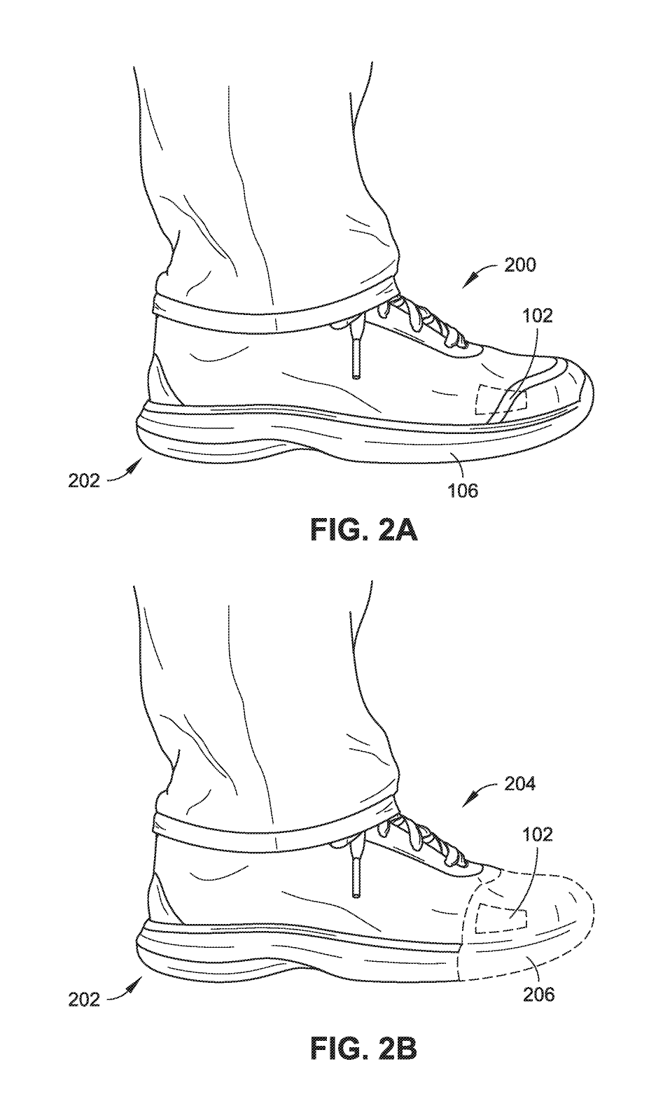

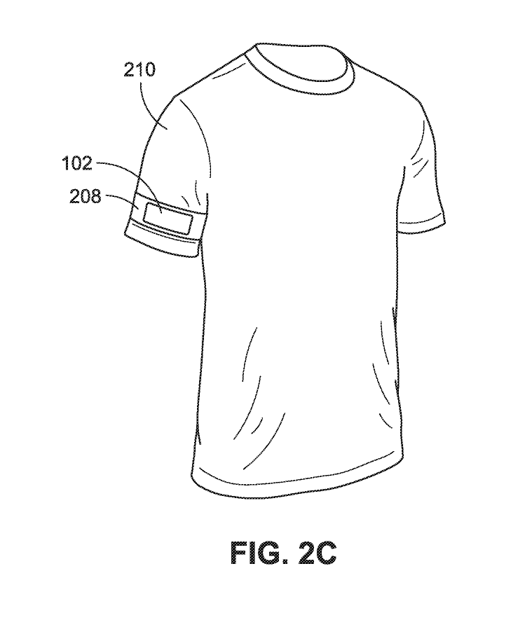

In embodiments, shown in FIG. 1, a system 100 is configured to monitor an environment through which an individual can move, where the system 100 can identify environmental objects in proximity to a body portion (e.g., appendage) of the individual and provide stimulation of a tissue of the individual remote from a site of impact between the individual and an environmental object or a site of impending impact between the individual and the environmental object. The system 100 includes a first device 102 and a second device 104. The first device 102 is configured to interface with a first body portion of the individual (shown in FIG. 1 as a foot), and the second device 104 is configured to interface with a second body portion of the individual (shown in FIG. 1 as a back of the individual). The first device 102 includes a first deformable substrate 106, a sensor assembly 108, and a reporter 110. The first deformable substrate 106 is configured to conform to the first body portion to provide support for the sensor assembly 108 and the reporter 110 relative to the individual. For example, the first deformable substrate 106 can comprise a deformable (e.g., conformable, flexible, stretchable, etc.) material configured to interface with, and conform to, the first body portion. The deformable and conformable nature of the first deformable substrate 106 facilitates interaction/interfacing with the first body portion, which includes a generally low-modulus and deformable natural skin surface. The first deformable substrate 106 can be associated with one or more of a shoe, a sock, a finger cot, a wrap, a glove, a ring, a bracelet, or any other wearable item, such that the first deformable substrate 106 can conform to respective human appendages. For example, the first body portion can include, but is not limited to, an arm, an elbow, a wrist, a hand, a finger, a leg, a knee, an ankle, a foot, or a toe. In embodiments, shown in FIG. 2A, the first deformable substrate 106 includes a footgear structure 200 that conforms to a foot 202 of the individual, such as to support the first device 102 and respective components (e.g., sensor assembly 108, reporter 110, etc.) relative to the foot 202. In embodiments, the first deformable substrate 106 is configured to couple to a footgear structure (e.g., shoe 204 shown in FIG. 2B), such as via an attachment or add-on structure 206 configured to couple to the footgear structure, such as to support the first device 102 and respective components (e.g., sensor assembly 108, reporter 110, etc.) relative to the foot 202. In embodiments, shown in FIG. 2C, the first deformable substrate 106 includes an apparel clip structure 208 configured to couple to apparel worn by the individual, such as to support the first device 102 and respective components (e.g., sensor assembly 108, reporter 110, etc.) relative to a body portion of the individual (shown in FIG. 2C as an arm 210). In embodiments, the system 100 includes a footgear structure, an ankle cuff structure, or an apparel clip, combinations thereof, configured to couple to the first deformable substrate 106. In embodiments, the first device 102 can include or employ epidermal electronics, where the first deformable substrate 106 can include one or more of a stretchable/flexible fabric, an elastomeric polymer, a hydrocolloid film, a nanomembrane (e.g., silicon nanomembrane), or other deformable/conformable material. The first deformable substrate 106 can be positioned in proximity with (conform to) the skin surface according to various mechanisms including, but not limited to, affixed to the skin via an adhesive material (e.g., resident on the skin surface), held in place by an external pressure, such as pressure provided by a material wrapped around or about a body portion (e.g., a fabric, a garment, a bandage, etc.), or so forth.

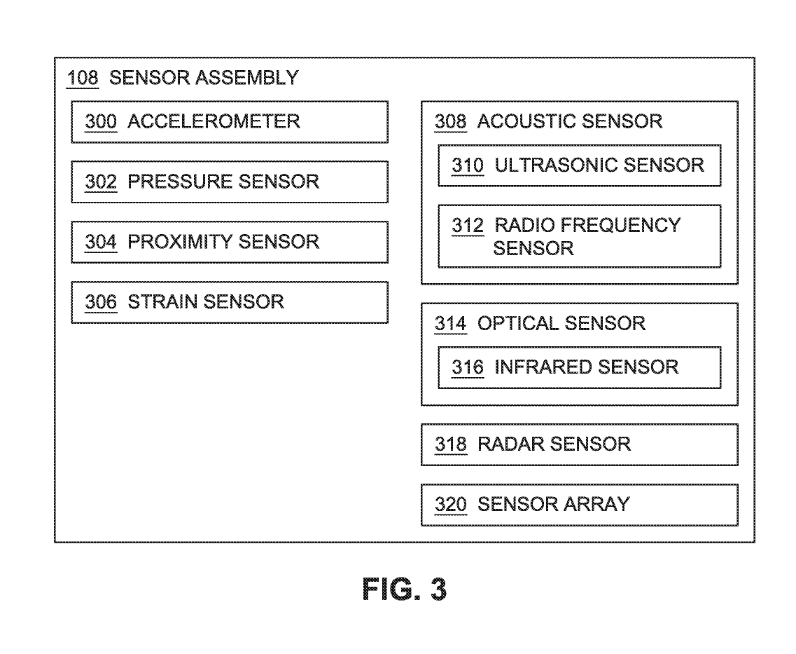

The sensor assembly 108 is coupled to the first deformable substrate 106 and includes a sensor configured to generate one or more sense signals associated with an impact between an environmental object and the first body portion or an impending impact between the environmental object and the first body portion. For example, the sensor assembly 108 can monitor the first body portion and/or regions proximate the first body portion to determine whether an environmental object is nearby, whether an environmental object is in a path of the first body portion (e.g., within a foot path during walking), whether an impact with an environmental object has occurred, whether an impact with an environmental object is likely to occur (e.g., via estimation of travel path, object movement, or the like), or other determination. In embodiments, shown in FIG. 3, the sensor assembly 108 includes an accelerometer 300, a pressure sensor 302, a proximity sensor 304, a strain sensor 306, an acoustic sensor 308 (e.g., an ultrasonic sensor 310, a radio frequency (RF) sensor 312), an optical sensor 314 (e.g., an infrared sensor 316), a radar sensor 318, a sensor array 320, or combinations thereof. In embodiments, the accelerometer 300 is configured to provide information representative of a movement of the first device 102, and, therefore, of a movement or gait of the individual and the first body portion to which the sensor assembly 108 is secured (e.g., via the first deformable substrate 106). The movement can be indicative of an impact to the first body portion with an environmental object. In embodiments, the accelerometer 300 is a microelectromechanical (MEMs) device.

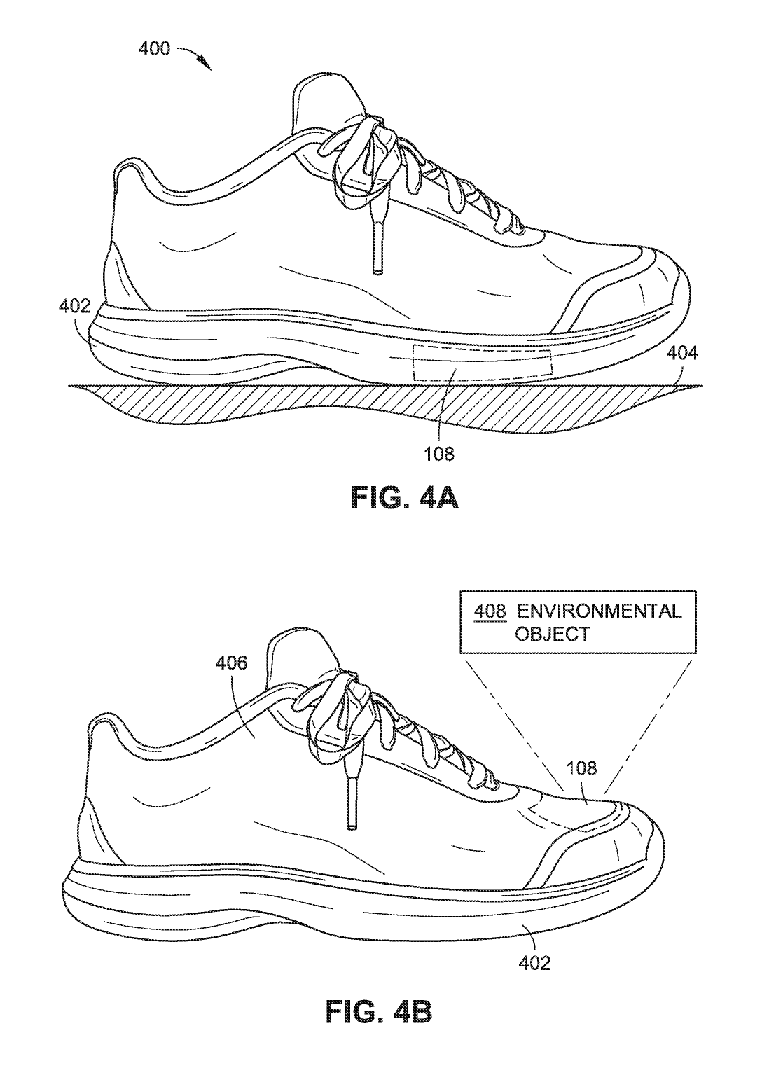

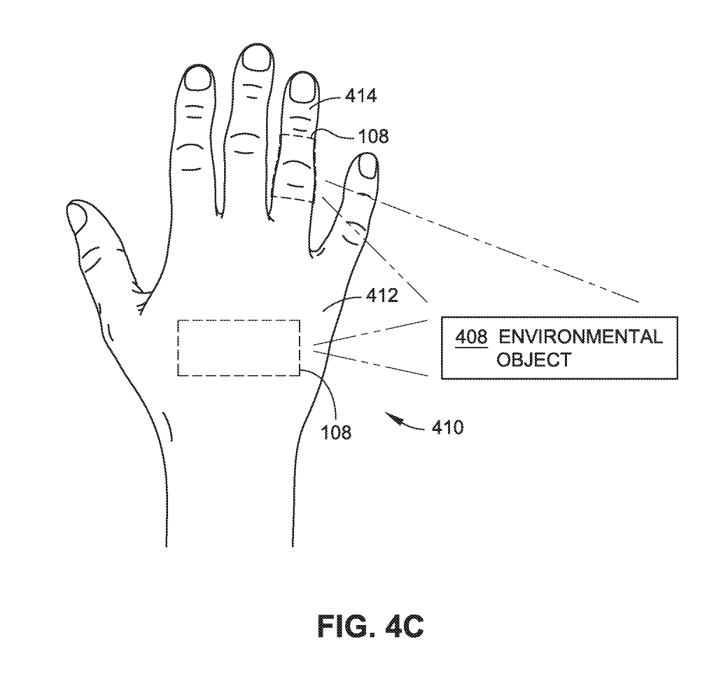

In embodiments, such as when the first body portion includes a foot or portion thereof, the sensor assembly 108 is configured for positioning proximate a sole of the foot or footgear structure to measure contact between the sole and a ground surface. For example, FIG. 4A shows a shoe 400 having a sole 402 with the sensor assembly 108 positioned proximate the sole 402 to measure contact between the sole 402 and a ground surface 404. The sensor assembly 108 can include, but is not limited to, the pressure sensor 302, the proximity sensor 304, the optical sensor 314, or combinations thereof. In embodiments, the pressure sensor 302 is configured to generate one or more output signals responsive to detection of a change in pressure, such as caused by contact of the sole 402 with the ground surface 404. In embodiments, the proximity sensor 302 is configured to generate one or more output signals responsive to detection of a change in pressure, such as caused by contact of the sole 402 with the ground surface 404. In embodiments, the proximity sensor 304 is configured to generate one or more output signals responsive to detection of a change in proximity between the sole 402 and the ground surface 404 or other object (e.g., an environmental object), such as caused by contact of the sole 402 with the ground surface 404 or impending contact when the sole 402 approaches the ground surface 404 during movement of the first body portion. In embodiments, the optical sensor 314 is configured to detect one or more optical signals and generate one or more sense signals in response thereto. The optical sensor 314 can detect and identify environmental objects (e.g., the ground surface 404) and their proximity relative to the shoe 400 or sole 404 based on the detected optical signals. In embodiments, the optical sensor 314 is configured to detect optical signals reflected from a surface of the environmental object. The origin of the optical signals can include the optical sensor 314, or other component of the system 100. In an embodiment, a processor (e.g., processor 116 of the first device 102, processor 116 of the second device 104, processor 116 of an external device, etc.) is configured to determine a proximity of the environmental object relative to the sensor assembly 108, an identity of the environmental object, or combinations thereof, based on the sense signals from the sensor assembly 108 (e.g., from the optical sensor 314).

In embodiments, such as when the first body portion includes a foot or portion thereof, the sensor assembly 108 is configured for positioning proximate an upper surface (e.g., top surface) of the foot or footgear structure to measure contact between the upper surface and an environmental object. For example, FIG. 4B shows the shoe 400 having the sole 402 and a shoe upper 406 configured to cover an upper surface (e.g., top surface), side surface, front surface, etc. of the foot. The sensor assembly 108 is coupled to the shoe upper 406 to measure contact between the foot (or the portion of the shoe 400 covering the foot) and an environmental object (e.g., environmental object 408). For example, the sensor assembly 108, or one or more sensors thereof, can be oriented to detect contact between the environmental object 408 and an upper region of the foot (or a top region of the shoe upper 406, such as shown in FIG. 4B), between the environmental object 408 and a side region of the foot (or side region of the shoe upper 406), between the environmental object 408 and a front region of the foot (e.g., toe) or a front region of the shoe upper 406, between the environmental object 408 and a back region of the foot (or a back region of the shoe upper 406), or combinations thereof. The sensor assembly 108 can include, but is not limited to, the pressure sensor 302, the proximity sensor 304, the optical sensor 314, or combinations thereof. In embodiments, the pressure sensor 302 is configured to generate one or more output signals responsive to detection of a change in pressure, such as caused by contact of the upper 406 with the environmental object 408. In embodiments, the proximity sensor 302 is configured to generate one or more output signals responsive to detection of a change in pressure, such as caused by contact of the upper 406 with the environmental object 408. In embodiments, the proximity sensor 304 is configured to generate one or more output signals responsive to detection of a change in proximity between the upper 406 with the environmental object 408, such as caused by contact of the upper 406 with the environmental object 408 or impending contact when the upper 406 approaches the environmental object 408 during movement of the first body portion (e.g., toe, heel, foot, etc.). In embodiments, the optical sensor 314 is configured to detect one or more optical signals and generate one or more sense signals in response thereto. The optical sensor 314 can detect and identify environmental objects (e.g., environmental object 408) and their proximity relative to the shoe 400 or upper 406 based on the detected optical signals. In embodiments, the optical sensor 314 is configured to detect optical signals reflected from a surface of the environmental object 408. The origin of the optical signals can include the optical sensor 314, or other component of the system 100.

In embodiments, such as when the first body portion includes a hand or portion thereof, the sensor assembly 108 is configured for positioning proximate a surface of the hand (e.g., palm, top of hand, inner surface of finger, outer surface of finger) to measure contact between the surface of the hand and an environmental object. For example, FIG. 4C shows a hand 410 with the sensor assembly 108 coupled (e.g., via the first deformable substrate 106) to one or more of a top surface 412 of the hand 410 or a finger 414 of the hand 410 to measure contact between the hand 410 and the environmental object 408 (e.g., user input device, such as a keyboard, keypad, graphical user interface, touchscreen, touchpad, desktop, tabletop, or other device or object). For example, the sensor assembly 108, or one or more sensors thereof, can be oriented to detect contact between the environmental object 408 and the finger 414 (or surface thereof), between the environmental object 408 and the top surface 412 of the hand 410, between the environmental object 408 and a palm surface of the hand 410, or combinations thereof. The sensor assembly 108 can include, but is not limited to, the pressure sensor 302, the proximity sensor 304, the optical sensor 314, or combinations thereof. In embodiments, the pressure sensor 302 is configured to generate one or more output signals responsive to detection of a change in pressure, such as caused by contact of the hand 410 (or portion thereof) with the environmental object 408. In embodiments, the proximity sensor 302 is configured to generate one or more output signals responsive to detection of a change in pressure, such as caused by contact of the hand 410 with the environmental object 408. In embodiments, the proximity sensor 304 is configured to generate one or more output signals responsive to detection of a change in proximity between the hand 410 with the environmental object 408, such as caused by contact of the hand 410 (or portion thereof, such as the finger 414) with the environmental object 408 or impending contact when the hand 410 approaches the environmental object 408 during movement of the individual. In embodiments, the optical sensor 314 is configured to detect one or more optical signals and generate one or more sense signals in response thereto. The optical sensor 314 can detect and identify environmental objects (e.g., environmental object 408) and their proximity relative to the hand 410 based on the detected optical signals. In embodiments, the optical sensor 314 is configured to detect optical signals reflected from a surface of the environmental object 408. The origin of the optical signals can include the optical sensor 314, or other component of the system 100.

In embodiments, the sensor assembly 108 is configured to detect a gait of the individual to which the system 100 is applied. For example, the output signals from one or more of the accelerometer 300, the pressure sensor 302, or the proximity sensor 304 can be analyzed by the processor 116 of the first device 102, the second device 104, an external device, or the like (e.g., via execution of one or more protocols to compare the output signals to reference data) to determine a gait of the individual. In embodiments, the sensor assembly 108 is configured to detect an environmental object in a path of the first body portion, an expected path of the first body portion, or combinations thereof. For example, the path or the expected path of the first body portion can include a path of travel of the first body portion, including but not limited to, a path of travel of a foot (e.g., during walk, running, etc.), a path of travel of a hand (e.g., during a typing motion, during a walking or running motion, during a grasping motion, etc.), or combinations thereof. The sensor assembly 108 can include, but is not limited to, the acoustic sensor 308, the ultrasound sensor 310, the radio frequency sensor 312, the optical sensor 314, the infrared sensor 316, the radar sensor 318, the sensor array 320, or combinations thereof, to detect the environmental object in the path or expected path of the first body portion. In embodiments, the acoustic sensor 308 is configured to detect one or more acoustic signals and generate one or more sense signals in response thereto. The acoustic signals can include, for example, ultrasonic signals (detectable by the ultrasonic sensor 310), radio frequency signals (detectable by the radio frequency sensor 312), other acoustic signals, or combinations thereof. The acoustic sensor 308 can detect and identify environmental objects (e.g., environmental object 408) and their proximity relative to a path of the first body portion based on the detected acoustic signals. For example, the acoustic sensor 308 can be oriented toward a direction of travel of the first body portion to detect acoustic signals from environmental objects in a path of travel of the first body portion. In embodiments, the acoustic sensor 308 is configured to detect acoustic signals reflected from a surface of the environmental object in a path of the first body portion. The origin of the acoustic signals can include the acoustic sensor 308, or other component of the system 100. In embodiments, the optical sensor 314 is configured to detect one or more optical signals and generate one or more sense signals in response thereto. The optical signals can include, for example, infrared signals (detectable by the infrared sensor 316), other optical signals, or combinations thereof. The optical sensor 314 can detect and identify environmental objects (e.g., environmental object 408) and their proximity relative to a path of the first body portion based on the detected optical signals. For example, the optical sensor 314 can be oriented toward a direction of travel of the first body portion to detect optical signals from environmental objects in a path of travel of the first body portion. In embodiments, the optical sensor 314 is configured to detect optical signals reflected from a surface of the environmental object in a path of the first body portion. The origin of the optical signals can include the optical sensor 314, or other component of the system 100. In embodiments, the radar sensor 318 is configured to detect one or more radar signals and generate one or more sense signals in response thereto. The radar signals can include, for example, electromagnetic signals in the radio wavelengths, electromagnetic signals in the microwave wavelengths, or combinations thereof. The radar sensor 318 can detect and identify environmental objects (e.g., environmental object 408) and their proximity relative to a path of the first body portion based on the detected radar signals. For example, the radar sensor 318 can be oriented toward a direction of travel of the first body portion to detect radar signals from environmental objects in a path of travel of the first body portion. In embodiments, the radar sensor 318 is configured to detect radar signals reflected from a surface of the environmental object in a path of the first body portion. The origin of the radar signals can include the radar sensor 318, or other component of the system 100. In embodiments, the sensor assembly 108 includes the sensor array 320 having a plurality of sensors. The positioning of the plurality of sensors of the sensor array 320 can facilitate detection of orientation and/or direction of the environmental object relative to the first body portion, such as during motion of the first body portion, during motion of the environmental object, or during motion of each of the first body portion and the environmental object. As such, by detecting the environmental object with the sensor array 320, the sensor array 320 can detect the environmental object in a path or expected path of the first body portion, can detect an orientation of the environmental object relative to the first body portion, or combinations thereof.

The reporter 110 of the first device 102 is operably coupled to the sensor assembly 108 and is configured to generate one or more communication signals responsive to activity by the sensor assembly 108. For example, in embodiments, the reporter 110 generates one or more communication signals responsive to generation of one or more sense signals by the sensor assembly 108 (e.g., sense signals associated with an impact or impending impact between the first body portion and an environmental object). The communication signals generated by the reporter 110 can be remotely transmitted, such as through wireless or wired communication protocols, to provide data to the second device 104, to a device or location external to the system 100, to a network external to the system 100, or combinations thereof. For example, the reporter 110 can include an antenna, a transceiver, another communication device, or combinations thereof to generate and remotely transmit the communication signals responsive to generation of the sense signals by the sensor assembly 108. In embodiments, the reporter 110 transmits the communication signals to the second device 104 to determine whether to stimulate the tissue remote from the first body portion and site of impact or site of potential impact. For example, the reporter 110 can transmit the communication signals to the second device 104, where the processor 116 can analyze the communication signals to determine whether to initiate or alter stimulation by the stimulator 118. For example, the communication signals can be associated with an impact between the first body portion and the environmental object, as detected by the sensor assembly 108, can be associated with an impending impact between the first body portion and the environmental object, as detected and/or monitored by the sensor assembly 108, or combinations thereof. In embodiments, at least a portion of the reporter 110 is coupled to the first deformable substrate 106 to support the reporter 110 relative to the first body portion of the individual.

In embodiments, shown in FIG. 1, the system 100 includes the second device 104 configured to interface with the second body portion, where such second body portion is remote from the first body portion (e.g., the body portion on which the first device 102 is positioned). For example, the first body portion (with which the first device 102 is configured to interact) can include a first portion of a foot (e.g., a toe, a sole, an upper surface), whereas the second body portion can include a second portion of the foot distinct from the first portion (e.g., a top portion of the foot when the first portion is a toe or sole). For instance, the second body portion can include a portion of the individual that is less likely to be affected by the nerve damage associated with neuropathy than the first portion, such that stimulation of a tissue proximate the second body portion can provide perceptible effects. In embodiments, the first body portion includes a foot and the second body portion includes an ankle (e.g., an ankle associated with the same leg as the foot with which the first device 102 is interacting, an ankle associated with a different leg as the foot with which the first device 102 is interacting, etc.). In embodiments, the first body portion includes a foot and the second body portion includes a leg (e.g., a leg associated with the same leg as the foot with which the first device 102 is interacting, a leg associated with a different leg as the foot with which the first device 102 is interacting, etc.). In embodiments, the first body portion includes a foot and the second body portion includes a back of the individual (e.g., an example of which is shown in FIG. 1).

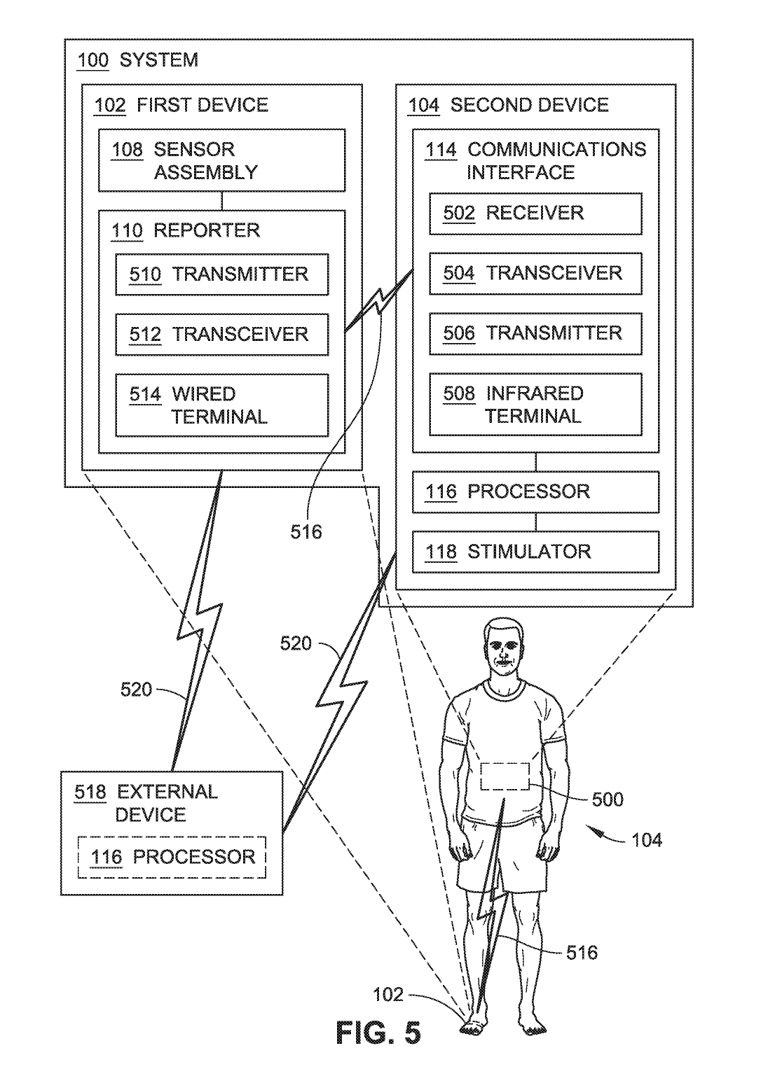

In embodiments, the second device 104 includes a second deformable substrate 112, a communications interface 114, a processor 116, and a stimulator 118. The second deformable substrate 112 is configured to conform to the second body portion to provide support for the communications interface 114, the processor 116, and the stimulator 118 relative to the individual. Alternative or additionally, one or more of the first device 102 or an external device (e.g., a mobile communications device) can include the processor 116 or additional processor 116 for providing analytical functionalities and/or instructions to other components of the system 100. For example, the second deformable substrate 112 can comprise a wearable component having a deformable (e.g., conformable, flexible, stretchable, etc.) material configured to interface with, and conform to, the second body portion. The deformable and conformable nature of the second deformable substrate 112 facilitates interaction/interfacing with the second body portion, which includes a generally low-modulus and deformable natural skin surface. The second deformable substrate 112 can be associated with one or more of a shoe, a sock, a finger cot, a wrap, a glove, a ring, or a bracelet such that the second deformable substrate 112 can conform to respective human appendages, upper back, lower back, or the like. For example, the second body portion can include, but is not limited to, an arm, an elbow, a wrist, a hand, a finger, a leg, a knee, an ankle, a foot, a toe, a torso, a back, a neck, or the like. In embodiments, shown in FIG. 5, the second deformable substrate 112 includes a patch 500 configured for resident placement on the second body portion. For example, the patch 500 can be positioned in proximity with the second body portion, or a skin surface thereof, according to various mechanisms including, but not limited to, affixed to the skin via an adhesive material, held in place by an external pressure, such as pressure provided by a material wrapped around or about the second body portion (e.g., a fabric, a garment, a bandage, etc.), or so forth. In embodiments, the patch 500 is configured to support the stimulator 118 relative to the second body portion, such as by including an aperture to position the stimulator 118 at least partially within the patch 500. In embodiments, the patch 500 comprises a deformable (e.g., conformable, flexible, stretchable, etc.) material configured to flex, stretch, or combinations thereof, in response to movement by the second body portion. In embodiments, the system 100 includes a footgear structure, an ankle cuff structure, or an apparel clip, combinations thereof, configured to couple to the second deformable substrate 112. In embodiments, the second device 104 can include or employ epidermal electronics, where the second deformable substrate 112 can include one or more of a stretchable/flexible fabric, an elastomeric polymer, a hydrocolloid film, a nanomembrane (e.g., silicon nanomembrane), or other deformable/conformable material that can be affixed to the second body portion, such to provide resident placement of the second device 104 on the skin surface.

The communications interface 114 of the second device 104 is coupled to the second deformable substrate 112 and is configured to receive communication signals from the first device 102. For example, the communications interface 114 can relay information associated with the communication signals from the first device 102 to other components of the second device 104, such as to the processor 116 for determination as to whether to activate the stimulator 118, whether to alter operation of the stimulator 118, or the like. In embodiments, shown in FIG. 5, the communications interface includes one or more of a receiver 502, a transceiver 504, a transmitter 506, or a wired terminal 508 to receive information, transmit information, or combinations thereof between the second device 104 and the first device 102 (e.g., via the reporter 110). In embodiments, the reporter 110 can include one or more of a transmitter 510, a transceiver 512, or a wired terminal 514 to transmit communication signals generated responsive to operation of the sensor assembly 108 to one or more of the receiver 502, the transceiver 504, the transmitter 506, or the wired terminal 508 of the second device 104 via one or more wireless or wired communication connections 516. For example, the communication connection 516 may be a connection over a wireless network (e.g., WiFi, Zigbee, Bluetooth, etc.), a wired interface (e.g., Ethernet, USB, Firewire, etc.), or other communications connection (e.g., infrared, optical, ultrasound, acoustic, etc.). In embodiments, the communications interface 114 is a wireless networking device or wired networking device which establishes communication connection 516 and transmits and/or receives data/signals through communications connection 516. For example, wired connections can facilitate transfer of information between the first device 102 and the second device 104 when the first body portion and the second body portion are spatially close, such as where the first body portion includes a foot of the individual and where the second body portion includes a separate portion of the foot, an ankle, a leg of the individual, or the like. Wireless connections can facilitate transfer of information across a range of distances between the first device 102 and the second device 104, such as where the first body portion includes a foot of the individual and where the second body portion includes a leg of the individual, a back of the individual, or the like.

In embodiments, an external device (e.g., external device 518 shown in FIG. 5) can communicate with one or more components of the system 100, such as through communication connections 520, which can include the wired or wireless protocols disclosed herein. For example, the external device 518 can include the processor 116 to provide remote analysis of information from the first device 102 and/or the second device 104 to determine when to transmit information from the first device 102 to the second device 104, when the initiate activation of the stimulator 118, whether to alter activation protocols of the stimulator 118, when to transmit or store data associated with operation of the system 100 or the components thereof, when to transmit or store data associated with an impact or impending impact, or the like. In embodiments, the external device 518 can include a memory accessible by the processor 116, a display (e.g., a graphical user interface (GUI), a touchscreen assembly (e.g., a capacitive touch screen), a liquid crystal display (LCD), a light-emitting diode (LED) display, a projection-based display, or the like), an audio device for broadcasting audio signals, a user interface, or combinations thereof. In embodiments, the external device 518 includes a communication device, such as one or more of a mobile communication device or a computer system including, but not limited to, a mobile platform, a mobile computing devices (e.g., hand-held portable computers, Personal Digital Assistants (PDAs), laptop computers, netbook computers, tablet computers, or so forth), mobile telephone devices (e.g., cellular telephones and smartphones), devices that include functionalities associated with smartphones and tablet computers (e.g., phablets), portable game devices, portable media players, multimedia devices, satellite navigation devices (e.g., Global Positioning System (GPS) navigation devices), e-book reader devices (eReaders), Smart Television (TV) devices, surface computing devices (e.g., table top computers), Personal Computer (PC) devices, and other devices that employ touch-based human interfaces. In embodiments, the system 100 interfaces with (e.g., via external device 518) or forms a part of a mobile platform, such as a mobile health platform. For example the system 100 can form a part of or communicate with (e.g., receive data from, transmit data to, or share data with) a mobile health platform.

In embodiments, the communications interface 114 is configured to send communication signals (to the first device 102, to the external device 518, to an external network, etc.) associated with an operation of the stimulator 118, a status of an impact between the first body portion and an environmental object, an impending impact between the first body portion and an environmental object, or combinations thereof. For example, the communications interface 114 can generate communication signals associated with the operation of the stimulator 118 or with a status of an impact or impending impact between the first body portion and an environmental object, and transmit the communication signals via the transmitter 506, such as in response to instruction from the processor 116. For example, the status can include an indication that an impact has occurred, the status can include an indication that an impending impact was avoided (e.g., the first body portion and the environmental object avoided impact), or the like.

In embodiments, the processor 116 is operably coupled to the communications interface 114 to receive and analyze the communication signals received by the communications interface 114 from the first device 102. Alternatively or additionally, the processor 116 is operably coupled to the reporter 110 to control operation of the reporter 110, to provide analysis of the sense signals from the sensor assembly 108 at the first device 102, or the like. Alternatively or additionally, the processor 116 is located external to the system, such as being associated with external device 518, to provide external analysis of the sense signals from the sensor assembly 108, to provide external processing of the communication signals from the communications interface 114, to provide instructions to one or more components of the first device 102, the second device 104, or combinations thereof, or the like. In embodiments, the processor 116 instructs the stimulator 118 to stimulate a tissue proximate the second body portion (e.g., responsive to analysis of the communication signals, of the sense signals, or combinations thereof). For example, the processor 116 can compare data associated with the communication signals with reference data indicative of an impact to determine when an impact occurs between the first body portion and an environmental object. The reference data indicative of an impact (or a comparison module having the reference data included) can be stored in a memory device for retrieval by the processor 116 for comparison to the one or more output signals to determine when an impact occurs. As another example, the processor 116 can compare data associated with the communication signals with reference data indicative of an impending impact to determine when an impact is likely to occur (e.g., within a standard deviation for probability of impact) between the first body portion and an environmental object. The reference data indicative of an impending impact (or a comparison module having the reference data included) can be stored in a memory device for retrieval by the processor 116 for comparison to the one or more output signals to determine when an impact is likely to occur. When the processor 116 determines that an impact has occurred or is likely to occur, the processor 116 can activate the stimulator 118 (e.g., via direct communications, via the communications interface 114, via communication connections 520, or the like) to provide the individual with a signal to indicate the impact or impending impact. In embodiments, the processor 116 includes components to process the communication signals from the reporter 110 (or to process the sense signals directly from the sensor assembly 108) and to provide instruction to one or more components of the second device 104, such as to activate one or more of the stimulator 118 to stimulate tissue proximate the second body portion, to activate the communications interface to transmit information to the first device 102 or to another location (e.g., external device, external network, etc.) or the like. For example, the processor 116 can include a microprocessor, a central processing unit (CPU), a digital signal processor (DSP), an application-specific integrated circuit (ASIC), a field programmable gate entry (FPGA), or the like, or any combinations thereof, and can include discrete digital or analog circuit elements or electronics, or combinations thereof. In one embodiment, the computing device includes one or more ASICs having a plurality of predefined logic components. In one embodiment, the computing device includes one or more FPGAs having a plurality of programmable logic commands. In embodiments, at least a portion of the processor 116 is coupled to the second deformable substrate 112.

In embodiments, the stimulator 118 of the second device 104 is operably coupled to the processor 116 and can generate stimulation (e.g., via transmission of signals, direct stimulation, etc.) of a tissue of the second body portion remote from a site of impact between the environmental object and the first body portion or a site of potential impact between the environmental object and the first body portion responsive to instruction by the processor 116. For example, the tissue to be stimulated can be remote from tissue associated with the site of impact or potential impact between the environmental object and the first body portion. For example, the tissue associated with the site of impact or impending impact can be proximate the first device 102 (e.g., which can be positioned on a foot of the individual), where the sensor assembly 108 can detect an impact between the foot and an environmental object (e.g., the leg of a table in the room), and the tissue to be stimulated can be remote from the site of impact or impending impact (e.g., on the back) and proximate to the second device. In embodiments, the tissue to be stimulated by the stimulator 118 can include, but is not limited to, a nerve, such as a specific nerve (e.g., an innervating nerve of a muscle, a nerve associated with the back, a nerve associated with the foot, a nerve associated with the hand, etc.), a cutaneous tissue or nerve, a targeted nerve, a targeted nerve tree, a targeted nerve bundle, a targeted nerve pathway, a proprioceptor, a peripheral nerve, or the like. For example, the tissue can include a relatively short nerve (e.g., a nerve associated with the back) that is less likely to be affected by peripheral neuropathy as compared to a tissue of the first body portion (e.g., a nerve associated with the foot). In embodiments, the reporter 110 of the first device 102 can transmit communication signals associated with the impact to the second device 104 via the communications interface 114, whereby the processor 116 can activate the stimulator 118 to generate stimulation of a tissue of the back of the individual, such as to provide an indication to the individual of the impact. Since the tissue of the back is remote from the foot, and remote from the site of impact with the environmental object, the individual may sense the tissue stimulation with tissue not affected by (or less affected by) neuropathy.

Stimulating the tissue can facilitate coordination of movement or interpretation of nervous signals, or lack thereof, by an individual subject, such as by assisting in information transfer where nerves may be damaged (e.g., via neuropathy, injury, illness, etc.), providing the individual subject a signal to retrain actions in response to various stimuli (e.g., via a trained response to alter an interpretation of a nerve signal), assisting in proprioception, or the like. For example, the stimulation of the tissue proximate second body portion by the stimulator 118 can be a signal for the individual to lift their foot higher (e.g., responsive to interaction between the foot and an environmental object, such as a stair, platform, etc.), to move their hand differently (e.g., responsive to interaction between the hand and a doorknob, desk surface, wall surface, etc.), or the like. For example, an individual can be trained through repeated stimulation by the stimulator 118 of the second body portion and manual movement of the first body that, when the particular stimulation occurs, the first body portion should be moved. Such training can employ muscle memory, for example. Additionally or alternatively, such coordination of movement or interpretation of nervous signals can include, rely on, or result in, reinnervation, neural regeneration, neural plasticity, or remapping of a tissue responsive to the stimulation. For example, stimulation of the tissue can include stimulation of a tissue (e.g., an injured tissue or diseased tissue) that includes a nervous tissue having undergone regeneration and/or reinnervation (e.g., regrowth or reorganization such as branching), and/or alteration in nerve connections or nerve circuitry, to compensate for nervous tissue functionally damaged as by injury or disease (e.g., diabetic neuropathy). For example, stimulation of the tissue can include stimulation of a tissue (e.g., an injured tissue or diseased tissue) that includes a nervous tissue functionally damaged (by injury or disease) such that the stimulation of the tissue over time drives regeneration, reinnervation (e.g., regrowth or reorganization such as branching), or alteration in nerve connections or nerve circuitry, to compensate for loss of function of the damaged nervous tissue. For example, stimulation of the tissue can include stimulation of a tissue of the central nervous system (e.g., a nervous tissue associated with the spinal cord). Operation of the stimulator 118 can facilitate a therapeutic regimen, for example as part of a physical therapy regimen, occupational therapy regimen, stroke therapy regimen, or other rehabilitation.

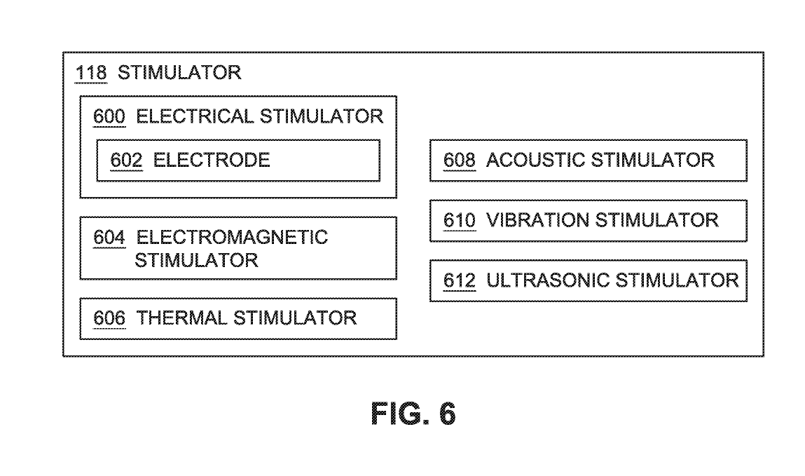

In embodiments, shown in FIG. 6, the stimulator 118 can include, but is not limited to, an electrical stimulator 600, one or more electrodes 602, an electromagnetic stimulator 604, a thermal stimulator 606, an acoustic stimulator 608, a vibration stimulator 610, or an ultrasonic stimulator 612. In embodiments, the stimulator 118 includes the electrical stimulator 600 having at least one electrode (e.g., electrode 602), where upon instruction by the processor 116, the electrical stimulator 600 generates electrical stimulation signals via the electrode to electrically stimulate a tissue proximate the second body portion. For example, the sensor assembly 108 can detect an impact between the first body portion and the environmental object (or the environmental object and the first body portion are nearing impact) and generate sense signals in response, where the reporter 110 generates communication signals responsive to the sense signals to communicate with the second device 104 proximate the tissue for stimulation. The processor 116 can then instruct the electrical stimulator 600 to generate electrical stimulation signals (e.g., via electrode 602) to stimulate the tissue, such as to provide the individual with electrical signaling indicative of an impact of their first body portion. For example, the electrical signaling can be perceptible by the individual (e.g., via nerve conduction at or proximate the tissue stimulated), whereas the impact of the first body portion with an environmental object may not be perceptible or as readily noticeable by the individual, such as due to impairment of nerve function (e.g., via peripheral neuropathy). The signaling can therefore alert the individual of an impact, an impending impact, etc., can train the individual to perform various responses to the signaling, such as adjusting gait, or the like.

In an embodiment, the stimulator 118 includes the electromagnetic stimulator 604, where the electromagnetic stimulator 604 stimulates a tissue proximate the second body portion. For example, the electromagnetic stimulator 604 can generate one or more electromagnetic stimulation signals (e.g., via an electromagnetic transducer, an electromagnetic actuator, etc.) to stimulate the tissue proximate the second body portion responsive to control by the processor 116. In an embodiment, the electromagnetic stimulator 604 is configured to deliver an electromagnetic excitatory stimulus to a tissue (e.g., a specific nerve (e.g., an innervating nerve of a muscle, a nerve associated with the back, a nerve associated with the foot, a nerve associated with the hand, etc.), a cutaneous tissue or nerve, a targeted nerve, a targeted nerve tree, a targeted nerve bundle, a targeted nerve pathway, a proprioceptor, a peripheral nerve, or the like) proximate the second body portion on which the second device 104 is positioned to provide electromagnetic signaling to the individual indicative of an impact to their first body portion. For example, the electromagnetic signaling can be perceptible by the individual (e.g., via nerve conduction at or proximate the tissue stimulated), whereas the impact of the first body portion with an environmental object may not be perceptible or as readily noticeable by the individual, such as due to impairment of nerve function (e.g., via peripheral neuropathy). The signaling can therefore alert the individual of an impact, an impending impact, etc., can train the individual to perform various responses to the signaling, such as adjusting gait, or the like.

In an embodiment, the stimulator 118 includes the thermal stimulator 606, where the thermal stimulator 606 stimulates a tissue proximate the second body portion. For example, the thermal stimulator 606 can generate one or more thermal stimulation signals (e.g., temperature changes, temperature gradients, etc.) to stimulate the tissue proximate the second body portion responsive to control by the processor 116. In an embodiment, the thermal stimulator 606 is configured to deliver a thermal excitatory stimulus to a tissue (e.g., a specific nerve (e.g., an innervating nerve of a muscle, a nerve associated with the back, a nerve associated with the foot, a nerve associated with the hand, etc.), a cutaneous tissue or nerve, a targeted nerve, a targeted nerve tree, a targeted nerve bundle, a targeted nerve pathway, a proprioceptor, a peripheral nerve, or the like) proximate the second body portion on which the second device 104 is positioned to provide thermal signaling to the individual indicative of an impact to their first body portion. For example, the thermal signaling can be perceptible by the individual (e.g., via nerve conduction at or proximate the tissue stimulated), whereas the impact of the first body portion with an environmental object may not be perceptible or as readily noticeable by the individual, such as due to impairment of nerve function (e.g., via peripheral neuropathy). The signaling can therefore alert the individual of an impact, an impending impact, etc., can train the individual to perform various responses to the signaling, such as adjusting gait, or the like.

In an embodiment, the stimulator 118 includes the acoustic stimulator 608, where the acoustic stimulator 608 stimulates a tissue proximate the second body portion. For example, the acoustic stimulator 608 can generate one or more acoustic stimulation signals (e.g., via an acoustic transducer, an acoustic actuator, etc.) to stimulate the tissue proximate the second body portion responsive to control by the processor 116. In an embodiment, the acoustic stimulator 606 is configured to deliver an acoustic excitatory stimulus to a tissue (e.g., a specific nerve (e.g., an innervating nerve of a muscle, a nerve associated with the back, a nerve associated with the foot, a nerve associated with the hand, etc.), a cutaneous tissue or nerve, a targeted nerve, a targeted nerve tree, a targeted nerve bundle, a targeted nerve pathway, a proprioceptor, a peripheral nerve, or the like) proximate the second body portion on which the second device 104 is positioned to provide acoustic signaling to the individual indicative of an impact to their first body portion. For example, the acoustic signaling can be perceptible by the individual (e.g., via nerve conduction at or proximate the tissue stimulated), whereas the impact of the first body portion with an environmental object may not be perceptible or as readily noticeable by the individual, such as due to impairment of nerve function (e.g., via peripheral neuropathy). The signaling can therefore alert the individual of an impact, an impending impact, etc., can train the individual to perform various responses to the signaling, such as adjusting gait, or the like.

In an embodiment, the stimulator 118 includes the vibration stimulator 610, where the vibration stimulator 610 stimulates a tissue proximate the second body portion. For example, the vibration stimulator 610 can generate one or more vibration stimulation signals (e.g., via a mechanical actuator) to stimulate the tissue proximate the second body portion responsive to control by the processor 116. In an embodiment, the vibration stimulator 610 is configured to deliver a vibration excitatory stimulus to a tissue (e.g., a specific nerve (e.g., an innervating nerve of a muscle, a nerve associated with the back, a nerve associated with the foot, a nerve associated with the hand, etc.), a cutaneous tissue or nerve, a targeted nerve, a targeted nerve tree, a targeted nerve bundle, a targeted nerve pathway, a proprioceptor, a peripheral nerve, or the like) proximate the second body portion on which the second device 104 is positioned to provide vibration signaling to the individual indicative of an impact to their first body portion. For example, the vibration signaling can be perceptible by the individual (e.g., via nerve conduction at or proximate the tissue stimulated, such as a cutaneous tissue), whereas the impact of the first body portion with an environmental object may not be perceptible or as readily noticeable by the individual, such as due to impairment of nerve function (e.g., via peripheral neuropathy). The signaling can therefore alert the individual of an impact, an impending impact, etc., can train the individual to perform various responses to the signaling, such as adjusting gait, or the like.

In an embodiment, the stimulator 118 includes the ultrasonic stimulator 612, where the ultrasonic stimulator 612 stimulates a tissue proximate the second body portion. For example, the ultrasonic stimulator 612 can generate one or more ultrasonic stimulation signals (e.g., via an ultrasonic transducer, an ultrasonic actuator, etc.) to stimulate the tissue proximate the second body portion responsive to control by the processor 116. In an embodiment, the ultrasonic stimulator 612 is configured to deliver an ultrasonic excitatory stimulus to a tissue (e.g., a specific nerve (e.g., an innervating nerve of a muscle, a nerve associated with the back, a nerve associated with the foot, a nerve associated with the hand, etc.), a cutaneous tissue or nerve, a targeted nerve, a targeted nerve tree, a targeted nerve bundle, a targeted nerve pathway, a proprioceptor, a peripheral nerve, or the like) proximate the second body portion on which the second device 104 is positioned to provide ultrasonic signaling to the individual indicative of an impact to their first body portion. For example, the ultrasonic signaling can be perceptible by the individual (e.g., via nerve conduction at or proximate the tissue stimulated), whereas the impact of the first body portion with an environmental object may not be perceptible or as readily noticeable by the individual, such as due to impairment of nerve function (e.g., via peripheral neuropathy). The signaling can therefore alert the individual of an impact, an impending impact, etc., can train the individual to perform various responses to the signaling, such as adjusting gait, or the like.

In embodiments, the stimulator 118 provides transdermal stimulation of a tissue of the second body portion. For example, the stimulator 118 (e.g., electrical stimulator 600, electromagnetic stimulator 604, thermal stimulator 606, acoustic stimulator 608, vibration stimulator 610, ultrasonic stimulator 612, or combinations thereof) can be configured to generate stimulation signals to provide subcutaneous signaling via transdermal stimulation by the stimulator 118 located proximate a skin surface of the individual (such as via coupling with the patch 500, embedding within the patch 500, etc.). In embodiments, the stimulator 118 is configured for at least partial implantation within the second body portion. For example, the second deformable substrate 112 can support one or more components of the second device 104 relative to the second body portion, while the stimulator 118 is partially or fully implanted within the second body portion (e.g., partially transdermal, partially subdermal, fully subdermal, etc.). The processor 116 can communicate with the stimulator 118 through wired communication protocols, wireless communication protocols, or combinations thereof, to activate, deactivate, or otherwise control operation of the stimulator 118 partially or fully implanted within the second body portion. In embodiments, the type of stimulator 118 (or multiple stimulators 118) utilized by the second device 104 depends on a location of the tissue to be stimulated, such as a depth of the particular tissue. For example, for cutaneous tissue, the stimulator 118 can include one or more of the vibration stimulator 610 configured to generate one or more vibration stimulation signals and to direct the one or more vibration stimulation signals to the cutaneous tissue or the thermal stimulator 606 configured to generate one or more thermal stimulation signals and to direct the one or more thermal stimulation signals to the cutaneous tissue. For example, for subcutaneous tissue, such as an innervating nerve of a muscle, the stimulator 118 can include the electrical stimulator 600 configured to generate one or more electric stimulation signals (e.g., via one or more electrodes 602) and to direct the one or more electric stimulation signals to the innervating nerve or other subcutaneous tissue.

In an embodiment, the stimulator 118 is configured to deliver a stimulus providing stochastic resonance to a tissue of the body part of the individual subject. For example, the stimulator 118 (e.g., the vibration stimulator 610) can stimulate (e.g., with mechanical or acoustic vibration) as a sub-sensory input that can enhance the response of the nervous system of the individual subject to other sensory inputs, such as the response of stimulation provided by one or more other stimulators 118 used to provide signaling to the tissue proximate the second body portion to alert of activity corresponding to the first body portion (e.g., impact, impending impact, etc.). For example, a stimulator (e.g., one or more stimulators 118) can apply vibratory stimuli to one portion of a muscle (e.g., back) or tendon to influence responsiveness of another portion of the muscle (e.g., spindles) or tendon (e.g., Golgi tendon organ). In an embodiment, the stimulator 118 can stimulate (e.g., with vibration, such as with vibration stimulator 610) to provide stochastic resonance to the stimulation for nerve signaling. For example, the system 100 can include a vibration effector (e.g., vibration stimulator 610) to provide Gaussian noise stimulation (e.g., at 0-300 Hz) to improve muscle activity and/or nerve transmission during signaling by one or more stimulators 118. For example, the system 100 can include a vibration effector (e.g., vibration stimulator 3914) in a shoe or boot worn by a person with decreased sensation in the toes, or in a glove, wrap, or sleeve worn by a person with decreased sensation in the fingers or hand. For example, a vibrating stimulator can provide stimulations at a similar frequency to that of the stimulator 118 signaling the tissue to improve response (e.g., to increase perception of the signaling). In an embodiment, the stimulator 118 is configured to deliver a stimulus to the body portion of the individual subject providing stochastic resonance to a distal tissue. For example, a stimulator (e.g., one or more stimulators 118) can apply vibratory stimuli to a proximal body portion (e.g., a wrist) to influence responsiveness of a laterally distal body portion (e.g., one or more fingers). For example, a stimulator (e.g., one or more stimulators 118) can apply vibratory stimuli to one portion of a body portion, such as the dorsal portion of a body portion (e.g., a top of a foot), to influence responsiveness of another portion of the body portion, such as the ventral portion of the body portion (e.g., the sole of the foot).

In an embodiment, the system 100 includes at least one flexible or stretchable electronic component. For example, at least one of the first device 102, the second device 104, the sensor assembly 108, the reporter 110, communications interface 114, the processor 116, the stimulator 118, or associated circuitry thereof, can include or be formed of flexible or stretchable electronics coupled to one or more of the first deformable substrate 106 or the second deformable substrate 112. For example, interconnects (not illustrated) between these components or within the circuitry can include or be formed of flexible or stretchable electronics (e.g., serpentine conducting tracings allowing for stretchable interconnects) and coupled to the one or more of the first deformable substrate 106 or the second deformable substrate 112. For example, a power source, can include or be formed of flexible or stretchable electronics and be coupled to one or more of the first deformable substrate 106 or the second deformable substrate 112 to supply power to components of the first device 102 and the second device 104. In an embodiment, the at least one flexible or stretchable electronic component includes at least one of a wavy, bent, mesh (e.g., open mesh), buckled, or serpentine geometry. In an embodiment, the at least one flexible or stretchable electronic component includes at least one nanowire, at least one nanoribbon, or at least one nanomembrane. For example, the system 100 can include one or more multifunctional electronic units comprising a stretchable/flexible system including a sensor assembly (e.g., sensor assembly 108), stimulator (e.g., stimulator 118), and power source in communication via associated circuitry (e.g., with reporter 110, communications interface 114, processor 116, etc.), including interconnects, residing in or on a deformable substrate (e.g., one or more of the first deformable substrate 106 or the second deformable substrate 112).

In an embodiment, the system 100 can include at least one ultrathin electronic component. For example, an ultrathin (e.g., less than 20 micrometers) electronic component can include a thinned wafer (e.g., thinned silicon wafer bonded to a polymer substrate), an ultrathin chip, or the like. For example, ultrathin circuitry can include conductive layers formed on a deformable substrate (e.g., one or more of the first deformable substrate 106 or the second deformable substrate 112) such as parylene by evaporation deposition with ultraviolet (UV) lithography and etching. For example, at least one of the first device 102, the second device 104, the sensor assembly 108, the reporter 110, communications interface 114, the processor 116, the stimulator 118, or associated circuitry thereof can include ultrathin electronics.

In an embodiment, the system 100 can include at least one electrically conductive thread, yarn, or textile. For example, at least one of the first device 102, the second device 104, the sensor assembly 108, the reporter 110, communications interface 114, the processor 116, the stimulator 118, or associated circuitry thereof can include at least one electrically conductive thread or yarn. Electrically conductive threads, yarns, or textiles can be configured to provide sufficient current to induce at least one of a wired or wireless coupling, e.g., between electronic components. For example, electronically conductive threads, yarns, or textiles may form the processor 116 (or circuitry thereof) or other circuitry configured to function in communication between the sensor assembly 108, one or more stimulators 118, or other circuitry of the system 100. For example, electronically conductive threads, yarns, or textiles may form at least a portion of circuitry configured to function in communication between a plurality of multifunctional electronic units each comprising a sensor assembly 108 and a reporter 110 or a communications interface 114, a processor 116, and a stimulator 118. Electrically conductive fibers, threads, and yarns can include a metallic material, semi-metallic material, sem i-insulative material, semi-conductive material (e.g., silicon and a gallium arsenide), or transparent conductive material (e.g., an indium-tin-oxide (ITO) material). Electrical threads or yarns can be embedded in textiles using weaving, knitting or embroidery, for example, or can be attached using nonwoven production techniques such as adhesion. For example, electrically conductive yarns having curved configuration can be attached to an elastic textile (e.g., by sewing or by adhesion) and can form all or part of a sensor assembly 108 that measures one or more physical characteristics of an individual, e.g., as the curved configuration is altered, such as due to particular skin topography or the like.

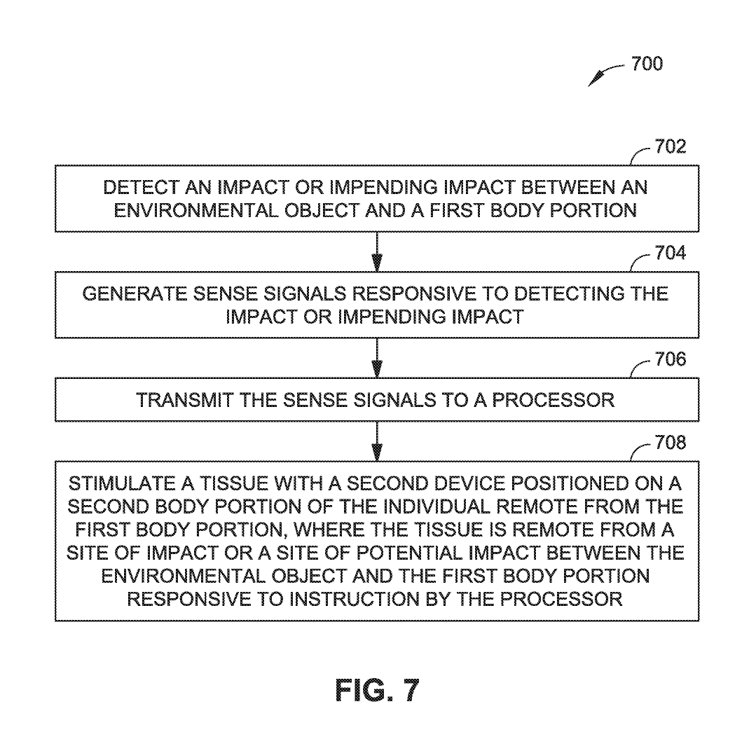

FIG. 7 illustrates a method 700 for monitoring or detecting an interaction between a first body portion of an individual subject and an environmental object and for stimulating a tissue of a second body portion remote from the first body portion or from a location of the interaction, which can result in increased perception by the individual subject of the interaction, particularly where the first body portion may have inhibited nerve function as compared to the second body portion (e.g., due to neuropathy, such as peripheral neuropathy). Method 700 shows detecting an impact or impending impact between an environmental object and a first body portion in block 702. For example, the sensor assembly 108 can detect an impact or impending impact between the first body portion (e.g., a foot, a toe, a hand, a finger, etc.) and an environmental object (e.g., a floor surface, a wall surface, a furniture surface, etc.). The sensor assembly 108 is part of the first device 102 configured for positioning on the first body portion, and thus the sensor assembly 108 can monitor and/or detect interactions between the first body portion and environmental objects around the first body portion. Method 700 also includes generating one or more sense signals responsive to detecting the impact or impending impact in block 704. For example, the sensor assembly 108 can generate one or more output signals responsive to detecting the impact or impending impact between the first body portion and the environmental object, such as via the accelerometer 300, the pressure sensor 302, the proximity sensor 304, the strain sensor 306, the acoustic sensor 308 (e.g., ultrasonic sensor 310, radio frequency (RF) sensor 312), the optical sensor 314 (e.g., infrared sensor 316), the radar sensor 318, the sensor array 320, or combinations thereof. Method 700 also includes transmitting the one or more sense signals to a processor in block 706. For example, the reporter 110 can receive the one or more output signals from the sensor assembly 108 and transmit communication signals, via wired protocol, wireless protocol, or combinations thereof, to the second device 104 via the communications interface 114. Method 700 further includes stimulating a tissue with a second device positioned on a second body portion of the individual remote from the first body portion, where the tissue is remote from a site of impact or a site of potential impact between the environmental object and the first body portion responsive to instruction by the processor in block 708. For example, the processor 116 can receive and analyze the sense signals from the sensor assembly 108, from the information received by the communications interface 114 from the reporter 110 of the first device 102, from the external device 518, or combinations thereof, to determine whether to activate the stimulator 118, and accordingly activate the stimulator, such as when an impact or impending impact has been detected.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations can include software or other control structures. Electronic circuitry, for example, may have one or more paths of electrical current constructed and arranged to implement various functions as described herein. In some implementations, one or more media can be configured to bear a device-detectable implementation when such media hold or transmit device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, the various embodiments described herein can be implemented, individually and/or collectively, by various types of electro-mechanical systems having a wide range of electrical components such as hardware, software, firmware, and/or virtually any combination thereof and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Examples of electro-mechanical systems include but are not limited to a variety of consumer electronics systems, medical devices, as well as other systems such as motorized transport systems, factory automation systems, security systems, and/or communication/computing systems. Electro-mechanical as used herein is not necessarily limited to a system that has both electrical and mechanical actuation except as context may dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

With respect to the use of substantially any plural and/or singular terms herein, the plural can be translated to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

This disclosure has been made with reference to various example embodiments. However, those skilled in the art will recognize that changes and modifications may be made to the embodiments without departing from the scope of the present disclosure. For example, various operational steps, as well as components for carrying out operational steps, may be implemented in alternate ways depending upon the particular application or in consideration of any number of cost functions associated with the operation of the system; e.g., one or more of the steps may be deleted, modified, or combined with other steps.

Additionally, as will be appreciated by one of ordinary skill in the art, principles of the present disclosure, including components, may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create a means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof are intended to cover a non-exclusive inclusion, such that a process, a method, an article, or an apparatus that comprises a list of elements does not include only those elements but may include other elements not expressly listed or inherent to such process, method, system, article, or apparatus.

In embodiments, the system is integrated in such a manner that the system operates as a unique system configured specifically for function of the system 100 used to monitor for interactions between an individual and body portions and signal a tissue in response thereto, and any associated computing devices of the system operate as specific use computers for purposes of the claimed system, and not general use computers. In embodiments, at least one associated computing device of the system operates as a specific use computer for purposes of the claimed system, and not a general use computer. In embodiments, at least one of the associated computing devices of the system is hardwired with a specific ROM to instruct the at least one computing device. In embodiments, one of skill in the art recognizes that the systems described herein (e.g., system 100) and associated systems/devices effect an improvement at least in the technological field of environmental sensing and tissue signaling.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system, comprising:
a first device configured to interface with a first body portion of an individual, the first device including
a first deformable substrate configured to conform to the first body portion;
a sensor assembly coupled to the first deformable substrate, the sensor assembly including a sensor configured to generate one or more sense signals associated with an impact between an environmental object and the first body portion or an impending impact between the environmental object and the first body portion; and
a reporter operably coupled to the sensor assembly and configured to generate one or more communication signals responsive to generation of the one or more sense signals by the sensor assembly for remote transmission, the one or more communication signals associated with the impact between the environmental object and the first body portion or the impending impact between the environmental object and the first body portion;
a processor operably coupled to at least one of the sensor assembly or the reporter to process at least one of the one or more sense signals or the one or more communication signals;
a second device configured to interface with a second body portion of the individual remote from the first body portion, the second device including
a second deformable substrate configured to conform to the second body portion;
a communications interface coupled to the second deformable substrate and configured to receive at least one of the one or more communication signals from the first device or instructions from the processor; and
a stimulator operably coupled to the processor and configured to generate stimulation of a tissue of the second body portion remote from a site of impact between the environmental object and the first body portion or a site of potential impact between the environmental object and the first body portion responsive to instruction by the processor.

2.-11. (canceled)

12. The system of claim 1, wherein the sensor is configured to detect the environmental object in a path of the first body portion or an expected path of the first body portion.

13.-19. (canceled)

20. The system of claim 1, wherein the sensor assembly includes a sensor array including a plurality of sensors.

21. (canceled)

22. The system of claim 20, wherein the sensor array is configured to detect an orientation of the environmental object relative to the first body portion.

23.-29. (canceled)

30. The system of claim 1, wherein the stimulator is configured to generate transdermal stimulation of a tissue of the second body portion.

31. The system of claim 1, wherein the second deformable substrate includes a patch configured for resident placement on the second body portion.

32.-34. (canceled)

35. The system of claim 1, wherein the stimulator is configured for at least partial implantation within the second body portion.

36. The system of claim 1, wherein the tissue is remote from tissue associated with the site of impact between the environmental object and the first body portion or the site of potential impact between the environmental object and the first body portion.

37. The system of claim 1, wherein the first body portion includes a first portion of a foot, and wherein the second body portion includes a second portion of the foot.

38.-45. (canceled)

46. The system of claim 1, wherein the tissue includes a nerve innervating a muscle, and wherein the stimulator includes an electrical stimulator configured to generate one or more electric stimulation signals and to direct the one or more electric stimulation signals to the innervating nerve.

47.-52. (canceled)

53. The system of claim 1, further including a reporter configured to indicate an operation of the stimulator or a status of the impact between the environmental object and the first body portion or the impending impact between the environmental object and the first body portion.

54.-115. (canceled)

116. The system of claim 1, wherein the first body portion is a foot, and wherein the sensor is configured for positioning proximate at least one of a sole of the foot to measure contact between the sole and a ground surface, an upper surface of the foot to measure contact between the upper surface and the environmental object, or a toe of the foot to measure contact between the toe and the environmental object.

117. The system of claim 1, wherein the sensor includes at least one of an accelerometer, a pressure sensor, a proximity sensor, or a strain sensor.

118. The system of claim 12, wherein the sensor includes at least one of an acoustic sensor, an ultrasonic sensor, a radio frequency sensor, an optical sensor, an infrared sensor, or a radar sensor.

119. The system of claim 1, wherein the stimulator includes at least one of an electrical stimulator configured to generate one or more electric stimulation signals and to direct the one or more electric stimulation signals to at least a portion of the second body portion, an electromagnetic stimulator configured to generate one or more electromagnetic stimulation signals and to direct the one or more electromagnetic stimulation signals to at least a portion of the second body portion, a thermal stimulator configured to generate one or more thermal stimulation signals and to direct the one or more thermal stimulation signals to at least a portion of the second body portion, an acoustic stimulator configured to generate one or more acoustic stimulation signals and to direct the one or more acoustic stimulation signals to at least a portion of the second body portion, a vibration stimulator configured to generate one or more vibration stimulation signals and to direct the one or more vibration stimulation signals to at least a portion of the second body portion, or an ultrasonic stimulator configured to generate one or more ultrasonic stimulation signals and to direct the one or more ultrasonic stimulation signals to at least a portion of the second body portion.

120. The system of claim 1, wherein the first body portion includes a foot, and wherein the second body portion includes at least one of an ankle of the individual, a leg of the individual, or a back of the individual.

121. The system of claim 1, wherein the tissue includes a cutaneous tissue, and wherein the stimulator includes at least one of a vibration stimulator configured to generate one or more vibration stimulation signals and to direct the one or more vibration stimulation signals to the cutaneous tissue or a thermal stimulator configured to generate one or more thermal stimulation signals and to direct the one or more thermal stimulation signals to the cutaneous tissue.

122. The system of claim 1, wherein the communications interface includes at least one of a terminal coupled to the first device via a wired connection to receive the one or more communication signals from the first device or at least one of a receiver or a transceiver wirelessly coupled with the first device to wirelessly receive the one or more communication signals from the first device.

123. The system of claim 53, wherein the status includes at least one of an indication that the impact occurred or an indication that the impending impact was avoided.

124. The system of claim 1, wherein the first deformable substrate includes at least one of a footgear structure, an ankle cuff structure, or an apparel clip structure, or is configured to conform to a skin surface of the first body portion.
